(19) United States
(12) Patent Application Publication
(10) Pub. No.: US 2011/0184294 A1
(43) Pub. Date: Jul. 28, 2011

(54) TUBE FOR A SPHYGMOMANOMETER CUFF, SPHYGMOMANOMETER CUFF AND PROCESS

(75) Inventor: Thomas GRASSL, Lubeck (DE)

(73) Assignee: Drager Medical GmbH, Lubeck (DE)

(21) Appl. No.: 12/957,458

(22) Filed: Dec. 1, 2010

(30) Foreign Application Priority Data
Jan. 22, 2010 (EP) 10 151 429.7

(51) Int. Cl. A61B 5/022

(52) U.S. Cl. 600/499

(57) ABSTRACT

A tube (100) is provided for a sphygmomanometer cuff (200) for measuring blood pressure on an extremity of a patient. The tube (100) has, in at least one segment thereof, a cross section that has at least two cross-sectional segments (1, 3) having different extents of curvature in both a first state of non-use and a second state of measurement. Further a sphygmomanometer cuff (200) is provided with the tube (100). A process is provided for manufacturing a sphygmomanometer cuff (200) and for using a tube (100). A process is also provided for measuring blood pressure.

### CROSS REFERENCE TO RELATED APPLICATIONS

This application claims the benefit of priority under 35 U.S.C. §119 of European Patent Application EP 10 151 429.7 filed Jan. 22, 2010, the entire contents of which are incorporated herein by reference.

### FIELD OF THE INVENTION

The present invention pertains to a tube for a sphygmomanometer cuff. The present invention pertains, furthermore, to a sphygmomanometer cuff according, to a process for manufacturing a sphygmomanometer cuff, as well as to a process for measuring the blood pressure and using a tube for a sphygmomanometer cuff.

### BACKGROUND OF THE INVENTION

Sphygmomanometer cuffs that have a tube for their inflation have been known from practice.

### SUMMARY OF THE INVENTION

One object of the present invention is to provide another tube for a sphygmomanometer cuff. In addition, a sphygmomanometer cuff as well as processes are provided.

According to the invention, a sphygmomanometer cuff tube is provided for a sphygmomanometer cuff for measuring blood pressure on an extremity of a patient. The tube comprises at least one cross sectional region with at least two cross-sectional segments having different extents of curvature.

The tube according to the present invention is suitable and intended as a tube of a sphygmomanometer cuff for measuring blood pressure on an extremity of a patient. It is preferably air-tight or gas-tight and elastic.

The tube according to the present invention has, in at least one of its sections, a cross section that has at least two cross-sectional segments that have different extents of curvature or different curvatures. A first cross-sectional segment has a different extent of curvature than an extent of curvature of a second cross-sectional segment.

The term “may be” or “may have,” etc., is used in all the following explanations as a synonym of “preferably is” or “preferably has.”

An “extremity of a patient” designates the arms and legs of the patient.

A “sphygmomanometer cuff” designates here an inflatable means carried by the physician, which is intended and designed to measure blood pressure in a non-invasive manner.

In an embodiment according to the present invention, the at least two cross-sectional segments having different extents of curvature or different curvatures have different extents of curvature or are curved differently in both a first state of non-use and in a second state of measurement.

The term “first state of non-use” as it is being used here designates a first state of the tube, in which the tube is being used for measurement and is also not placed on the extremity. The state of non-use may be a state of the tube in which the tube is not subject to any actions of external forces and/or deformations.

The term “second state of measurement” as it is being used here designates a second state of the tube, in which the tube is used to measure blood pressure, and in which especially air or a gas in general is admitted into its interior. The second state may describe a point in time at which the systolic or diastolic pressure is measured by means of the sphygmomanometer cuff.

In certain embodiments of the present invention, the tube may have the same cross section with the features according to the present invention described above continuously or over its entire length. This can make it advantageously possible, for example, to manufacture the tube as a yard ware, which is favorable in terms of manufacture and cost-effectiveness.

In other embodiments of the present invention, the tube has a cross section with the above-described features according to the present invention in one segment only.

Such a segment may be an end segment of the tube. The segment of the tube may be intended to be connected to the sphygmomanometer cuff. The segment of the tube may be connected to the sphygmomanometer cuff.

The segment is a cuff connection in one embodiment according to the present invention.

The entire tube is a cuff connection in one embodiment according to the present invention.

The cuff connection is a separate connection piece in one embodiment according to the present invention. The tube according to the present invention, which is designed as a cuff connection, may be able to be connected or may be connected to another tube to prepare a sphygmomanometer cuff. The cuff connection may be connected to the tube according to the present invention or to another tube in a non-positive manner and/or in a positive-locking manner and/or by a connection in substance.

The term “cross section” as it is being used here designates in an embodiment according to the present invention an extension in space of the tube or of the tube material in a direction directed at right angles to, i.e., essentially at an angle of essentially or exactly 90° to a longitudinal direction of the stretched tube.

In one embodiment according to the present invention, the tube according to the present invention has, besides the above-described cross section, another cross section that is different therefrom. Ratios of curvature that are different from that in the above-described cross section may occur in the other cross section. The other cross section may be, in particular, round.

The term “cross-sectional segment” as it is being used here designates a sphygmomanometer cuff of the tube according to the present invention with a cross section as described above.

In another embodiment, the tube according to the present invention has, besides the two cross-sectional segments having different curvatures, other cross-sectional segments, which may in turn have any desired curvatures of their own.

The term “curvature” as it is being used here designates a deviation of the extension of a line or of a (curved) surface from a straight line or from a plane. It shall be borne in mind in this connection that a straight line also has a curvature (with a radius of 0) in the sense of the present invention.

A curvature is also defined according to the present invention as a bend. For example, a cross section in the shape of an equiangular triangle has two cross-sectional segments with different curvatures in the sense of the present invention: the angles between the sides (wherein the angles do not differ from each other due to the triangle being an equiangular triangle) and the curvature of the sides themselves, wherein the curvature of said sides themselves is defined, as was stated above, as a curvature having a radius of 0 based on their straight extension in the sense of the present invention. A cross section in the form of an isosceles triangle could consequently have cross-sectional segments with two or correspondingly more than two curvatures in the sense of the present invention.

The different curvatures of the individual cross-sectional segments or cross-sectional segments being considered may be relative to the curvatures of the outer surface and/or the inner surfaces of the tube at the level of the cross section being considered.

The term “having different curvature” as it is being used here means that at least two cross-sectional segments of the cross section of the tube have different curvatures and are especially curved to different extents.

In one embodiment of the present invention, a cross-sectional segment of the tube facing the extremity during the measurement has a curvature essentially equaling or equaling 0 degrees or a concave curvature—the latter being considered based on it being later in contact with an extremity—from a center of the extremity—in the first state of non-use.

A cross-sectional segment with a curvature of 0 degrees is a straight cross-sectional segment of the tube.

In one embodiment according to the present invention, the tube has a closed, essentially or approximately semicircular cross section in the first state of non-use. Such a cross section is shown, for example, in FIG. 1 attached.

In such an embodiment, a cross-sectional segment of the tube has a straight form, i.e., a curvature of essentially 0 degrees or exactly 0 degrees, and another has an arc-shaped or sickle-shaped form with a curvature differing from 0 degrees.

In another embodiment, a cross-sectional segment of the tube facing the extremity has a convex curvature, as it is shown, for example, in FIG. 2, relative to the extremity of the patient, with which this cross-sectional segment shall come into contact at a later point in time for the purpose of measurement, in the first state of non-use.

In another embodiment of the tube according to the present invention, the cross-sectional segment of the tube facing the extremity has a curvature of essentially equaling or equaling 0 degrees or a concave curvature relative to the extremity in the second state of measurement.

The term “concave curvature relative to the extremity” as it is being used here designates a curvature of one cross-sectional segment of the tube, which said curvature faces the extremity, such that the concavely curved segment more or less nestles against the extremity, contrary to a curvature that is convex relative to the extremity.

In one embodiment of the present invention, the concave curvature has a radius r in a range of r from and including 0.5 cm up to and including 2 cm in the second state of measurement. The center and hence the starting point for the determination of the radius is in an extremity and not outside same.

For example, a curvature, calculated according to the formula I/r, may have values ranging from and including 0.5 cm⁻¹ up to and including 2 cm⁻¹.

In another embodiment of the present invention the tube has a sickle-shaped, closed cross section relative to the extremity in the second state of measurement.

The term “sickle-shaped cross section” as it is being used here means that both cross-sectional segments of the cross section have a concave shape relative to the extremity. Such a sickle-shaped appearance of the cross section of the tube can be found, for example, in attached FIG. 2 of the drawings.

In another embodiment of the tube according to the present invention, a cross-sectional segment facing the extremity of the patient in the second state of blood pressure measurement has a concave curvature in the range of 0 to 1/r in an outer circumference, wherein r is the radius of an osculating circle placed in the area of a segment of the outer circumference of the extremity.

The term “osculating circle” as it is being used here designates a circle or circle of curvature that is placed in the area of a section of the outer circumference of the extremity and comes closest to a point on the cross-sectional segment tube facing the extremity.

The osculating circle placed in the area of a segment of the outer circumference may vary depending on the nature, for example, the position or curvature of an immediate or closer environment of the point on the cross-sectional segment of the tube facing the extremity. Thus, it may correspondingly have, in particular, different radii and/or circle centers, i.e., centers of curvature.

Since the curvature is considered in this embodiment at discrete points on the cross-sectional segment of the tube facing the extremity, discrete values for the curvature may vary greatly. For example, the cross-sectional segment of the tube facing the extremity may possibly have locally a convex curvature.

The entire curvature of the cross-sectional segment of the tube facing the extremity may be obtained by forming the integral of all the discrete curvatures present in the area of the cross-sectional segment of the tube facing the extremity.

The tube according to the present invention is manufactured in one embodiment according to the present invention from a thermoplastic or has such a material. In one embodiment according to the present invention, the tube according to the present invention has a polyurethane or consists thereof. In one embodiment according to the present invention, the tube according to the present invention is manufactured from a thermoplastic polyurethane, especially TPR 60 ShA (Shore A test method). However, other thermoplastic elastomers, PVC or silicone are also imaginable and are covered by the present invention.

In certain embodiments of the present invention, the tube according to the present invention has an external diameter smaller than 8 mm, preferably 5 mm, in at least one segment thereof or over its entire length.

The term “external diameter” as it is being used here may pertain in some embodiments according to the present invention to an overall diameter of an essentially symmetrical tube, such as a round shape or contour of the tube. The term may pertain to the maximum or average extension in a cross sectional direction.

In another embodiment of the present invention, the tube has at least one cuff connection or is designed as such. The cuff connection is in the interior of the sphygmomanometer cuff at least in some segments or completely and is subject to the pressure increase caused by the sphygmomanometer cuff.

The object of the present invention is accomplished, furthermore, by a sphygmomanometer cuff according to FIG. 1. All the advantages that can be gained with the tube according to the present invention can also be gained to the full extent with the sphygmomanometer cuff according to the present invention.

The sphygmomanometer cuff according to the present invention has at least one tube according to the present invention.

The tube may be connected to the sphygmomanometer cuff in at least some segments in a non-positive manner and/or in a positive-locking manner and/or by a connection in substance.

The sphygmomanometer cuff according to the present invention may have at least a first film and a second film as well as at least one tube according to the present invention, which is welded in between the first film and the second film of the sphygmomanometer cuff at least in some segments, in the area of the cuff connection.

The films arranged on the inside and/or on the outside of the sphygmomanometer cuff may be made of PU (polyurethane) or PVC or have polyurethanes at least in some segments.

On the inside, the sphygmomanometer cuff may have, furthermore, at least one velour coated in at least some segments. The inside may be in contact with the extremity of the patient during the second state of measurement.

Aside from the tube used, the sphygmomanometer cuff may be a conventional sphygmomanometer cuff, as it is known to a person skilled in the art for measuring the blood pressure of a patient.

The sphygmomanometer cuff according to the present invention can be manufactured by the tube according to the present invention being arranged, especially in a cuff connection thereof, between the film on the inside and the films on the outside of the sphygmomanometer cuff and connected to the films in a non-positive manner and/or in a positive-locking manner and/or by connection in substance.

For example, the tube may be connected to and/or between the two films in the area of the cuff connection by means of a high-frequency welding method (HF welding method).

In the area of the inside of the sphygmomanometer cuff, the cuff connection of the tube may have a straight surface, i.e., a curvature of essentially or exactly 0 degrees, in the first state of non-use.

The object of the present invention is accomplished, furthermore, by a process according to the present invention. All the advantages that can be gained with the tube according to the present invention can also be gained to the full extent with the processes according to the present invention.

A sphygmomanometer cuff according to the present invention is manufactured here by using at least one tube according to the present invention.

Furthermore, the tube being described here in a sphygmomanometer cuff is also an aspect of the present invention.

Furthermore, the measurement of the blood pressure of a patient, especially of a baby (newborn) by means of the sphygmomanometer cuff is another aspect of the present invention.

The present invention provides a tube for a sphygmomanometer cuff, which tube can advantageously ensure good transmission of the measured signal.

The cross-sectional segment facing the extremity is straight or has a concavely arched surface during the measurement. It can thus be advantageously ensured that the tube or its cuff connection does not lead to measurement artifacts above all during measurements on the arm of a newborn with an inherently very small diameter or it must be removed and reapplied after the measurement artifacts have been recognized for the purpose of a correct measurement. In addition, punctiform action of the sphygmomanometer cuff during the measurement can be advantageously avoided.

It may be advantageously possible with the tube according to the present invention to avoid an unfavorable ratio of the tube diameter to the diameters of the artery and extremity during the measurement of the blood pressure, as it can be observed above all during measurement on premature babies or newborns. This unfavorable ratio may lead, as the inventor could determine, to compression of the artery of the upper arm even by the pressure applied by the tube on the artery and to the artery not being compressed, as is required during blood pressure measurement by means of a sphygmomanometer cuff, by the pressure of the sphygmomanometer cuff itself, in case of a relatively small application surface of the tube during measurement, e.g., on the upper arm of a newborn. Since the application surface of the tube on the upper arm and hence also on the main artery thereof is markedly smaller with the use of the tube according to the present invention because of the geometric design thereof, the above-described unfavorable area ratio is not present. The measurement results obtained are therefore more reliable than those obtained with sphygmomanometer cuffs whose tubes have a cross-sectional profile different from that of the tube according to the present invention.

Due to its special cross-sectional profile as described above, the tube according to the present invention can be attached to the sphygmomanometer cuff such that at least segments thereof can also be present in the interior of the sphygmomanometer cuff. Kinking of the tube, which may occur in tubes according to the state of the art, which do not extend into the interior of the sphygmomanometer cuff, can be advantageously avoided hereby.

In addition, it may be advantageously possible to prevent the measured signal from being damped.

Furthermore, it is advantageously possible in some embodiments according to the present invention to reduce the pressure applied locally on the arm, above all by the tube, such that pressure sites or other adverse effects on tissues will not develop. Narrowing of the extremity in some segments can be advantageously avoided.

Furthermore, it may be possible with the use of the tube according to the present invention to also position the cuff connection directly at the beginning of the sphygmomanometer cuff without stiffening of the cuff occurring on a relatively large area, as it happens in solutions according to the state of the art, in which tube stems are welded as 90° angles on the outside of the sphygmomanometer cuff. It may in turn be advantageously possible in this manner to prevent a reduction of the cross section of the arm necessary for the measurement above all in newborns and/or to prevent a deformation of said cross section because of an increase in stiffness.

The present invention will be described below as an example with reference to the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a cross sectional view of a tube according to the present invention in a first state of non-use according to a first embodiment;

FIG. 2 is a cross sectional view of a tube according to the present invention in a first state of non-use according to a second embodiment;

FIG. 3 is a cross sectional view of a tube according to the present invention in a second state of measurement;

FIG. 4 is a cross sectional view through a sphygmomanometer cuff according to the present invention during a second state of measurement on an extremity;

FIG. 5 is a top view of a sphygmomanometer cuff according to the present invention in a first state of non-use;

FIG. 6 is a cross sectional view of a tube according to the present invention in a first state of non-use according to a third embodiment; and

FIG. 7 is a cross sectional view of a tube according to the present invention in a first state of non-use according to a fourth embodiment.

### DESCRIPTION OF THE PREFERRED EMBODIMENTS

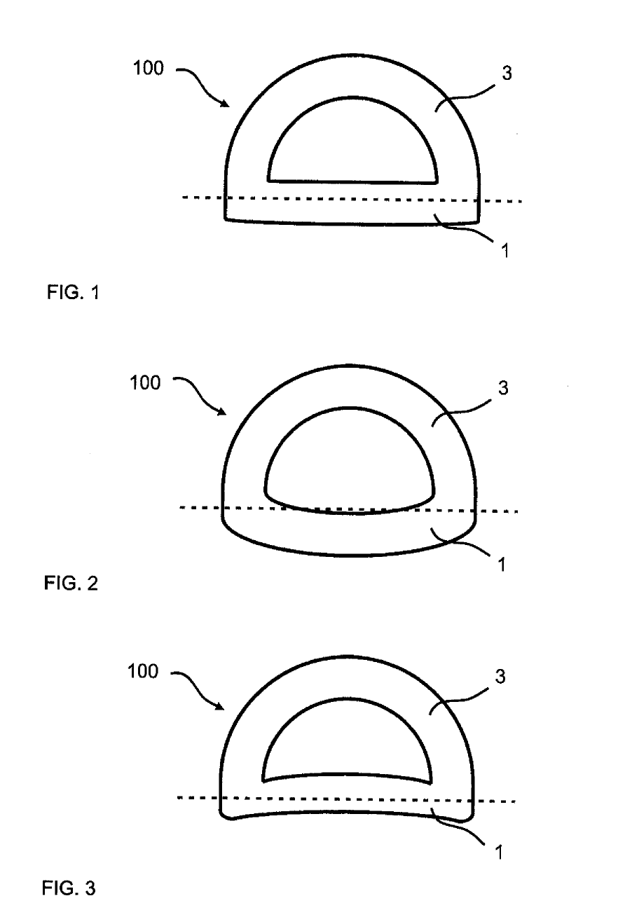

Referring to the drawings in particular, FIG. 1 shows a cross section of a tube 100 according to the present invention in a first state of non-use according to a first embodiment.

Tube 100 has a cross-sectional segment 1 facing the extremity (not shown in FIG. 1) (during its later use) and a cross-sectional segment 3 facing away from the extremity, which together represent a closed semicircle.

The cross-sectional segment 1 facing the extremity has a curvature of essentially 0 degrees. The cross-sectional segment 3 facing away from the extremity has a concave shape relative to the extremity on which the tube is to come to lie during the measuring operation.

The facing and facing-away cross-sectional segments 1, 3 thus have—compared to one another—different extents of curvature.

FIG. 2 shows a cross section of a tube according to the present invention in a first state of non-use according to a second embodiment.

The cross-sectional segment 1 facing the extremity has a (slightly) convex curvature relative to the extremity.

The facing-away and facing cross-sectional segments 1, 3 are thus curved again to different extents compared to one another.

FIG. 3 shows a cross section of a tube according to the present invention in a second state of measurement.

The cross-sectional segment 1 facing the extremity has a concave curvature relative to the extremity. The cross-sectional segment 3 facing away from the extremity likewise has a concave shape relative to the extremity.

The closed cross section of tube 100 thus has a concave, generally sickle-shaped form or contour relative to the extremity.

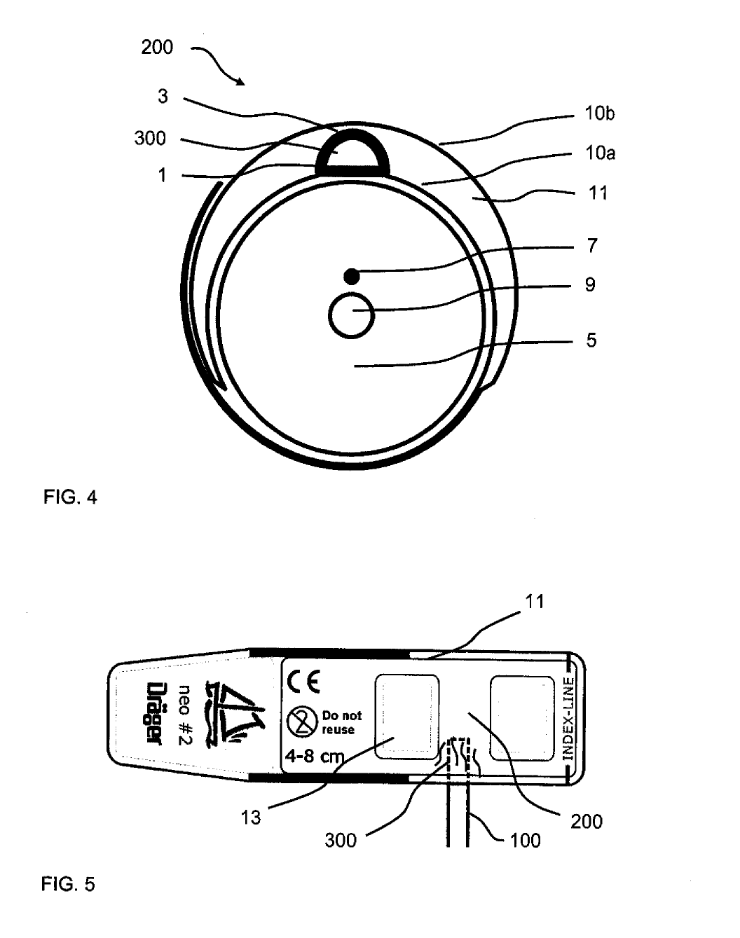

FIG. 4 shows a cross section through a sphygmomanometer cuff 200 according to the present invention during a second state of measurement on an extremity.

The sphygmomanometer cuff 200 is placed in a closed form around the extremity, e.g., an arm 5 with an artery 7 and with an upper arm bone 9.

The sphygmomanometer cuff 200 has the tube 100 according to the present invention designed as a cuff connection 300. As was discussed above, the tube 100 according to the present invention may optionally have a cuff connection 300 having the features according to the present invention of the cross-sectional segments 1, 3 having different extents of curvature, or cross-sectional segments 1, 3 having different extents of curvature may be provided continuously, i.e., not only in the area of its cuff connection 300.

As is shown in FIG. 4 (and also in FIG. 5), cuff connection 300 is arranged in an end area of the sphygmomanometer cuff 200.

As can be clearly recognized in FIG. 4, the cross section of cuff connection 300 has a sickle-shaped form or contour during the second state of measurement. Both the cross-sectional segment 1 of the cuff connection 300 facing the arm 5 and the cross-sectional segment 3 of the cuff connection 300 facing away from the arm 5 have a concave curvature relative to the arm 5. An external diameter of the cuff connection 300, i.e., an arch of the cross-sectional segment 1 facing the arm 5, may have, for example, a radius of 0.5 cm to 2 cm.

The sphygmomanometer cuff 200 according to the present invention can be manufactured by inserting the cuff connection 300 between two films 10a and 10b and welding it to same, e.g., by means of a high-frequency welding method. The two films 10a, 10b form an interior 11 (not visible in FIG. 5), to which pressure can be admitted to measure the blood pressure.

One of the cross-sectional segments 1, 3 or both cross-sectional segments 1, 3 and/or other cross-sectional segments of the tube according to the present invention or of the cuff connection 300 may also be alternatively a part of the film 10a and/or 10b, i.e., an integrated part thereof.

The cross-sectional segments 1 and 3 define a gas-tight interior 11 of the sphygmomanometer cuff 200. The artery 7 is compressed by means of the interior 11 during the measurement of the blood pressure. The interior 11 is provided here in the sphygmomanometer cuff 200 such that after it has been placed on the extremity, it is typically not in contact over the entire surface of said extremity. The interior 11, which can also be called a bubble, correspondingly occupies approximately half of the length of the sphygmomanometer cuff 200, as this can be recognized from FIG. 5, in case of great variations between different sphygmomanometer cuffs.

FIG. 5 shows a top view of the sphygmomanometer cuff 200 according to the present invention in a first state of non-use.

The first state of non-use may be, e.g., a state in which the sphygmomanometer cuff 200, as it is shown in FIG. 5, lies rolled out flat in front of an observer.

Cuff connection 300, which is located in the interior 11 of the sphygmomanometer cuff 200, is not visible in the view shown in FIG. 5. However, its position within the interior 11 is indicated by the folds shown in FIG. 5 as well as by the dash-dotted view.

At another end, the right-hand end relative to the view in FIG. 5, a Velcro fastener 13 is provided, which is used to close the sphygmomanometer cuff 200 around the extremity.

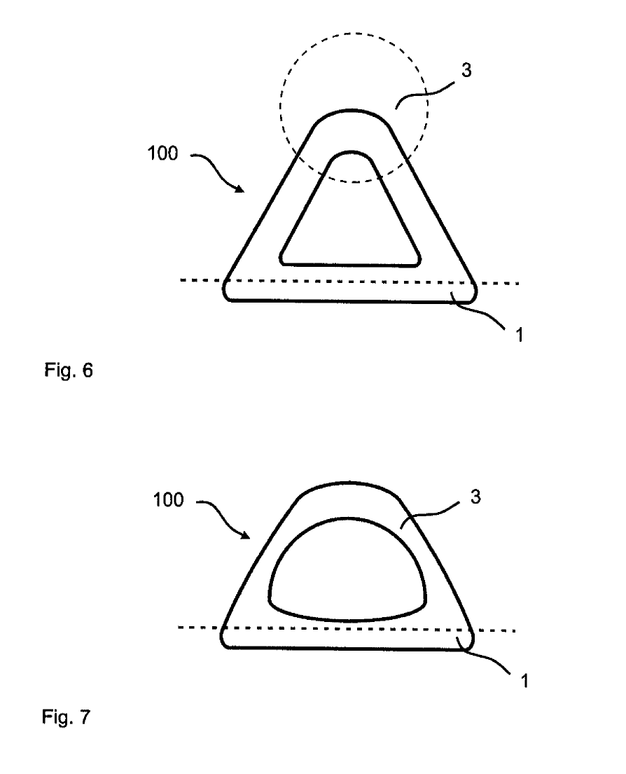

FIG. 6 shows a cross section of a tube according to the present invention in a first state of non-use according to a third embodiment, in which the cross section is equilateral or essentially equilateral. A more or less triangular shape of a cross section of a tube according to the present invention in a first state of non-use is shown in FIG. 7 for a fourth embodiment.

While specific embodiments of the invention have been described in detail to illustrate the application of the principles of the invention, it will be understood that the invention may be embodied otherwise without departing from such principles.

### APPENDIX

### List of Reference Numbers

100 Tube
200 Sphygmomanometer cuff
300 Cuff connection
1 Cross-sectional segment (facing the extremity)
3 Cross-sectional segment (facing away from the extremity)
7 Artery
9 Upper arm bone
10a Film
10b Film
11 Interior of the sphygmomanometer cuff
13 Velcro fastener

What is claimed is:

1. A sphygmomanometer cuff tube for a sphygmomanometer cuff for measuring blood pressure on an extremity of a patient, the tube comprising:
at least one cross sectional region with at least two cross-sectional segments having different extents of curvature.

2. A sphygmomanometer cuff tube in accordance with claim 1, wherein the cross-sectional segments have different extents of curvature in both a first state of non-use and a second state of measurement.

3. A sphygmomanometer cuff tube in accordance with claim 1, wherein said at least two cross-sectional segments include a cross-sectional segment facing the extremity during the measurement having a curvature essentially equaling 0 degrees or exactly 0 degrees or a concave curvature relative to the extremity in one or both a first state of non-use and a second state of measurement.

4. A sphygmomanometer cuff tube in accordance with claim 1, wherein the tube has a closed, essentially semicircular cross section in a first state of non-use.

5. A sphygmomanometer cuff tube in accordance with claim 1, wherein the at least one cross sectional region has at least one of a triangular or polygonal shape, an equilateral shape or an equiangular shape.

6. A sphygmomanometer cuff tube in accordance with claim 1, wherein:
the tube has a first state of non-use and a second state of measurement;
one of the at least two cross-sectional segments has a concave curvature in the second state of measurement with a radius r in a range of r from and including 0.5 cm up to and including 2 cm with a center in an area of the extremity.

7. A sphygmomanometer cuff tube in accordance with claim 1, wherein:
the tube has a first state of non-use and a second state of measurement;
the at least one cross sectional region has a sickle-shaped, closed cross section relative to the extremity in the second state of measurement.

8. A sphygmomanometer cuff tube in accordance with claim 1, wherein:
the tube has a first state of non-use and a second state of measurement;
one of the at least two cross-sectional segments facing an outer circumference of the extremity of the patient in the second state of measurement has a concave curvature facing the extremity in a range from 0 to 1/r, wherein r is the radius of an osculating circle placed in an area of a segment of an outer circumference of the extremity.

9. A sphygmomanometer cuff tube in accordance with claim 1, further comprising segments made of TPR 60 ShA or including TPR 60 ShA.

10. A sphygmomanometer cuff tube in accordance with claim 1, wherein the tube forms a cuff connection arranged at least in some segments or partially or fully in an interior of a sphygmomanometer cuff for measuring the blood pressure.

11. A sphygmomanometer cuff tube in accordance with claim 10, wherein
the cuff connection includes a cuff structure defining an interior to which pressure can be admitted to measure the blood pressure; and
the at least one cross sectional region is arranged at least partially or fully in the interior of the cuff structure.

12. A sphygmomanometer cuff comprising:
a cuff structure defining an interior to which pressure can be admitted to measure blood pressure;
at least one tube connected to the cuff structure and having a tube interior that may be in fluid communication with the interior, the tube having at least one cross sectional region with a first cross-sectional segment and a second cross-sectional segment, said first cross-sectional segment having a different extent of curvature than an extent of curvature of said second cross-sectional segment.

13. A sphygmomanometer cuff in accordance with claim 12, wherein the cuff structure comprises:
at least a first film and a second film, the at least one tube being welded in between the first film and the second film in at least some segments in an area of a cuff connection.

14. A sphygmomanometer cuff in accordance with claim 13, wherein:
the at least one tube forms a cuff connection with the at least one cross sectional region arranged at least in some segments or partially or fully in the interior of the cuff structure.

15. A sphygmomanometer cuff in accordance with claim 12, wherein said first cross-sectional segment and said second cross-sectional segment include a cross-sectional segment facing the extremity during the measurement having a curvature essentially equaling 0 degrees or a concave curvature relative to the extremity in one or both a first state of non-use and a second state of measurement.

16. A sphygmomanometer cuff in accordance with claim 12, wherein:
the at least one tube has a first state of non-use and a second state of measurement;
one of said first cross-sectional segment and said second cross-sectional segment facing an outer circumference of the extremity of the patient in the second state of measurement has a concave curvature facing the extremity in a range from 0 to 1/r, wherein r is a radius of an osculating circle placed in an area of a segment of an outer circumference of the extremity.

17. A process for manufacturing a sphygmomanometer cuff, the process comprising the steps of:
providing at least a first film and a second film;
connecting at least one tube to the at least said first film and said second film in an area of a cuff connection, the at least one tube having at least one cross sectional region with at least two cross-sectional segments having different extents of curvature.

18. A process for measuring blood pressure of a patient, the process comprising the step of:
providing a cuff structure defining an interior to which pressure can be admitted to measure the blood pressure;
providing at least one tube connected to the cuff structure and having a tube interior that may be in fluid communication with the interior, the tube having at least one cross sectional region with a first cross-sectional segment and a second cross-sectional segment, said first cross-sectional segment having a different extent of curvature than an extent of curvature of said second cross-sectional segment;
applying the cuff structure to a patient; and
regulating the pressure in the interior to measure the blood pressure of the patient.

19. A process for measuring blood pressure of a patient according to claim 18, wherein:
the at least one tube forms a cuff connection with at least one cross sectional region arranged at least in some segments or partially or fully in the interior of the cuff structure.

20. A process for measuring blood pressure of a patient according to claim 15, wherein:
the patient is a neonate.
